(19) United States
(12) Patent Application Publication
(10) Pub. No.: US 2022/0285035 A1
(43) Pub. Date: Sep. 8, 2022

(54) DEVICE AND METHOD OF PREDICTING DISEASE BY USING ELDERLY COHORT DATA

(71) Applicants: ELECTRONICS AND TELECOMMUNICATIONS RESEARCH INSTITUTE, Daejeon (KR); KOREA RESEARCH INSTITUTE OF STANDARDS AND SCIENCE, Daejeon (KR)

(72) Inventors: Jae Hak YU, Daejeon (KR); Se Jin PARK, Daejeon (KR); Soon Hyun KWON, Daejeon (KR); Nae Soo KIM, Daejeon (KR); Da Mee KIM, Daejeon (KR); Cheol Sig PYO, Daejeon (KR); Seung Hee Hong, Daejeon (KR)

(21) Appl. No.: 17/689,654

(22) Filed: Mar. 8, 2022

(30) Foreign Application Priority Data
Mar. 8, 2021 (KR) 10-2021-0030228
Jun. 22, 2021 (KR) 10-2021-0081013

(51) Int. Cl. G16H 50/70; G16H 50/20; G16H 50/30; A61B 5/00

(52) U.S. Cl. CPC G16H 50/70; G16H 50/20; G16H 50/30; A61B 5/7275

(57) ABSTRACT

The present invention relates to a device and method of predicting disease by using elderly cohort data, and more particularly, to a device and method of predicting disease by using elderly cohort data and an elderly disease prediction model applied thereto, which may predict an outbreak possibility of an elderly disease including cerebral stroke by using cohort data of 60 or more-year-old persons.

### CROSS-REFERENCE TO RELATED APPLICATIONS

This application claims priority under 35 U.S.C. § 119 to Korean Patent Application Nos. 10-2021-0030288, filed on Mar. 8, 2021, and 10-2021-0081013, Jun. 22, 2021, the disclosure of which is incorporated herein by reference in its entirety.

### TECHNICAL FIELD

The present invention relates to a device and method of predicting disease by using elderly cohort data, and more particularly, to a device and method of predicting disease by using elderly cohort data and an elderly disease prediction model applied thereto, which may predict an outbreak possibility of an elderly disease including cerebral stroke by using cohort data of 60 or more-year-old persons.

### BACKGROUND

Based on the statistics of cause of death provided from National Statistical Office in 2018, it has been reported that the total number of dead persons is 298,820, the number of dead men is 161,187, and the number of dead women is 137,633. Based on each cause of death in the statistics, it has been reported that malignant neoplasm (cancer) is 79,153 in number of patients, a heart disease is 32,004 in number of patients, pneumonia is 23,280 in number of patients, and a cerebrovascular disease is 22,940 in number of patients. Here, in a 60 or more-year-old patient group, a death rate caused by a heart disease and a cerebrovascular disease included in a circulatory disease is progressively increasing.

An elderly disease including the heat disease and the cerebrovascular disease has various symptoms and is variously classified, and due to this, is difficult to reliably evaluate a disorder caused by a corresponding symptom and a neurological damage accompanied thereby. Also, in patients having a past outbreak history, a possibility to re-outbreak is high, and thus, it is desperately required to develop technology which help to continuously trace and observe target persons to enable a patient to be diagnosed and cured at an appropriate time.

For example, cerebral stroke is one of main diseases which cause a function disorder of adults and elderly persons and is one of fatal diseases which cause difficulty in social or economic activities, on the basis of the degree of disorder. The cerebral stroke may variously occur based on the degree of disorder of patients or an accompanies disease, and thus, a current disorder level should be accurately evaluated and a risk factor should be continuously managed for each person.

In National Institutes of Health, national institutes of health stroke scale (NIHSS), which is widely used in quantitative measurement on a disorder after the outbreak of cerebral stroke, is globally and widely being used as an indicator where reliability and validity between inspection and re-inspection have been verified. The NIHSS is being widely used to overall evaluate a disorder of each cerebral stroke patient, but has a drawback which it is unable to provide an accurate prediction information result for evaluating an initial disorder.

### SUMMARY

Accordingly, the present invention provides a device and method of predicting disease by using elderly cohort data and an elderly disease prediction model applied thereto, which analyze cohort data of an elderly group defined as 60 or more-year-old persons by using a prediction model based on a convolution neural network (CNN) to predict the outbreak of an elderly disease, thereby providing objective diagnosis and a cure for elderly diseases.

The objects of the present invention are not limited to the aforesaid, but other objects not described herein will be clearly understood by those skilled in the art from descriptions below.

In one general aspect, a method of predicting disease by using elderly cohort data includes: collecting cohort data of an elderly group; preprocessing the collected cohort data; extracting an attribute in the collected cohort data and selecting a subset corresponding to the extracted attribute; and analyzing a degree of risk of a disease on the basis of the selected attribute set by using a disease prediction model.

In another general aspect, a device for predicting disease by using elderly cohort data includes: a data collector configured to collect cohort data of an elderly group; a data preprocessor configured to preprocess the collected cohort data; a subset selector configured to extract an attribute in the collected cohort data and select a subset corresponding to the extracted attribute; and a disease analyzer configured to analyze a degree of risk of a disease on the basis of the selected attribute set by using a disease prediction model.

A computer program according to another embodiment of the present invention for solving the above-described problem may be coupled to a computer which is hardware, may execute a method of predicting disease by using elderly cohort data, and may be stored in a computer-readable recording medium.

Other features and aspects will be apparent from the following detailed description, the drawings, and the claims.

### BRIEF DESCRIPTION OF THE DRAWINGS

FIG. 1 is a block diagram illustrating a configuration of a device for predicting disease by using elderly cohort data according to the present invention.

FIGS. 2 and 3 are reference tables for describing a process of constructing a data mart by using cohort data according to an embodiment of the present invention.

FIGS. 4A to 4C are reference diagram for describing a disease prediction model based on a 1D CNN according to the present invention.

FIGS. 5 and 6 are reference tables showing an element-based analysis result of a disease prediction model according to an embodiment of the present invention.

FIG. 7 is a flowchart for describing a process of predicting a disease by using elderly cohort data according to the present invention.

### DETAILED DESCRIPTION OF EMBODIMENTS

Embodiments of the present invention are provided so that this disclosure will be thorough and complete, and will fully convey the concept of the present invention to one of ordinary skill in the art. Since the present invention may have diverse modified embodiments, preferred embodiments are illustrated in the drawings and are described in the detailed description of the present invention. However, this does not limit the present invention within specific embodiments and it should be understood that the present invention covers all the modifications, equivalents, and replacements within the idea and technical scope of the present invention. In describing the present invention, a detailed description of known techniques associated with the present invention unnecessarily obscure the gist of the present invention, it is determined that the detailed description thereof will be omitted.

Moreover, each of terms such as “ . . . part”, “ . . . unit”, and “module” described in specification denotes an element for performing at least one function or operation, and may be implemented in hardware, software or the combination of hardware and software.

In the following description, the technical terms are used only for explain a specific exemplary embodiment while not limiting the present invention. The terms of a singular form may include plural forms unless referred to the contrary. The meaning of ‘comprise’, ‘include’, or ‘have’ specifies a property, a region, a fixed number, a step, a process, an element and/or a component but does not exclude other properties, regions, fixed numbers, steps, processes, elements and/or components.

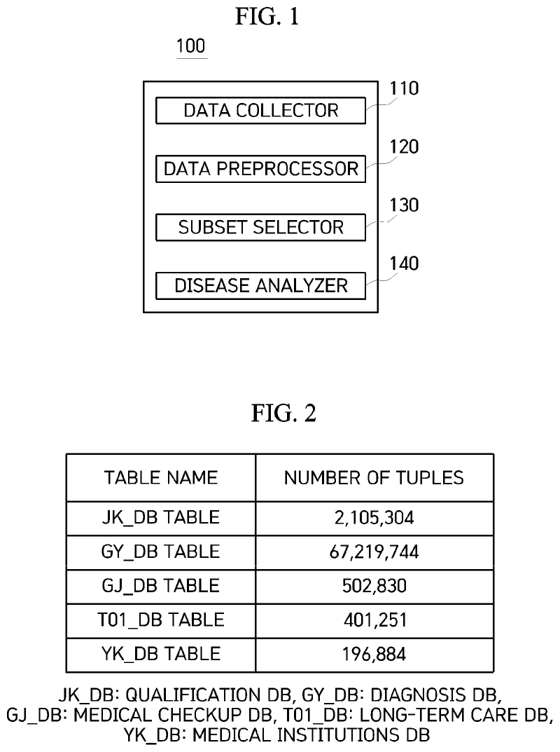

FIG. 1 is a block diagram illustrating a configuration of a device for predicting disease by using elderly cohort data according to the present invention.

Referring to FIG. 1, a device for predicting disease (hereinafter referred to as a disease prediction device) 100 by using elderly cohort data according to the present invention may include a data collector 110, a data preprocessor 120, a subset selector 130, and a disease analyzer 140. Here, elements included in the disease prediction device 100 may be for performing an essential function or operation in the present invention and may be added or modified according to additional embodiments or depending on the case.

The data collector 110 may collect cohort data of an elderly group.

Here, the cohort data of the elderly group may be collected in a research database which is built for research support for elderly persons such as prognosis analysis and risk factors of elderly diseases, and for example, a cohort database provided from institution such as National Health Insurance Service may correspond thereto. Also, the cohort data of the elderly group may include social and economic information, disorder and death information, medical use information include cure and health information, medical cure institution situation information, long-term elderly care service application, and use information, which include medical treatment and medical checkup.

In an embodiment, the data collector 110 may periodically update the cohort data stored in the database, and thus, may allow the disease prediction model to previously learn the updated cohort data. In detail, the disease prediction device 100 according to the present invention may calculate an outbreak rate (a risk degree) of a disease to be analyzed by using the disease prediction model receiving the cohort data, and thus, it may be needed to periodically update the database storing the cohort data.

The data preprocessor 120 may preprocess the collected cohort data.

In detail, in order to perform classification or prediction based on machine learning and deep learning, it may be needed to perform a preprocessing operation on raw data where a possibility of including pieces of repeated data, which are not complete and are inconsistent, is high. In the present invention, a preprocessing operation may be performed on the cohort data so as to improve and enhance the performance and accuracy of the disease prediction model.

In an embodiment, the data preprocessor 120 may remove a repeated tuple and a noise tuple in each data table included in the cohort data and may convert and normalize a data format so as to enable analysis through the disease prediction model. Here, the tuple may denote a record or a row in the data table.

Moreover, the data preprocessor 120 may generate a main data table associated with a disease which is to be predicted and may construct a data mart including a data table associated with a main disease code of the disease which is to be predicted, on the basis of joining of the generated main data tables.

FIGS. 2 and 3 are reference tables for describing a process of constructing a data mart by using cohort data according to an embodiment of the present invention.

When a prediction target disease according to the present invention is cerebral stroke, as in FIG. 2, a main data table relevant to cerebral stroke in which preprocessing of collected cohort data is reflected and the number of tuples corresponding to the main data table may be calculated. Subsequently, in order to calculate only data corresponding to 160 to 169 which are main disease codes associated with cerebral stroke, a data mart may be constructed by joining the main data table and a relevant data table like joined data table and the number of tuples shown in a table of FIG. 3, and the disease prediction model may perform analysis by using data of the constructed data mart.

The subset selector 130 may extract an attribute in the collected cohort data and may select a subset corresponding to the extracted attribute. For example, when a prediction target disease according to the present invention is cerebral stroke, the subset selector 130 may extract total 64 attributes in the cohort data. Here, the extracted attribute may include a continuity attribute, including a body mass index, proteinuria, total cholesterol level, serum creatinine level, and gamma GPT level, and a discrete attribute including daily drinking amount, smoking, the presence of hepatitis B antigen (HBeAg), and high-strength physical activity.

In an embodiment, the subset selector 130 may perform Z-score normalization based on the following Equation 1 on the attribute extracted from the collected cohort data.

$$\vec{{x}_{i}} = \frac{{x}_{i} - μ}{σ} \times α \qquad [ Equation 1 ]$$

Here, may denote each attribute, σ may denote a standard deviation of x, μ may denote an average of x, and α may denote a weight value.

Such a normalization process may convert data so that corresponding data is within a small range of 0.0 to 1.0, and thus, each attribute may have the same weight value. Therefore, like serum creatinine level in the extracted attribute, a range of a value may be wide, and thus, a case where the value depends on a measurement unit may be prevented.

Moreover, the subset selector 130 may calculate and select a subset where a probability distribution calculated in a case which uses all attributes extracted from the cohort data and a similar probability distribution are calculated, in performing data classification. Here, in order to calculate and select the subset, the subset selector 130 may use Hall's theorem. In detail, an entropy corresponding to Y including a best first search value and an attribute value and a condition probability based on Pearson's correlation coefficient between attributes and a target class may be calculated by using Hall's theorem. Also, the entropy corresponding to an arbitrary attribute Y may be calculated as the following Equation 2, in order to obtain an information profit of each attribute.

$$H ( Y ) = - {\sum }_{y ∈ Y}^{} p ( y ) {log}_{2} ( p ( y ) ) \qquad [ Equation 2 ]$$

Moreover, the subset selector 130 may evaluate a subset, where a largest value is calculated as a result of the calculation based on the following Equation 3, as a subset where an expression rate of all attributes is highest, and the disease prediction model may be analyzed by using a subset evaluated as a subset where an expression rate is highest. The following Equation 3 may represent a merit function for evaluating the degree to which all attributes of each subset (Fₐ⊂F) are efficiently expressed.

$$Merit ( {F}_{S} ) = \frac{k \overline{{r}_{cf}}}{\sqrt{k + k ( k - 1 ) \overline{{r}_{ff}}}} \qquad [ Equation 3 ]$$

Here, Fₛ may denote a subset, k may denote the number of attributes of Fₛ, r̅_̅{̅c̅f̅}̅ may denote an average distribution of attributes included in Fₛ, and r̅_̅{̅f̅f̅}̅ may denote an average correlation value of all attributes.

The disease analyzer 140 may analyze the degree of risk of a disease by using an attribute set selected through the disease prediction model. Here, the disease prediction model may be constructed as a disease prediction model based on a 1D CNN. Hereinafter, a detailed structure of the disease prediction model and an analysis result based thereon will be described.

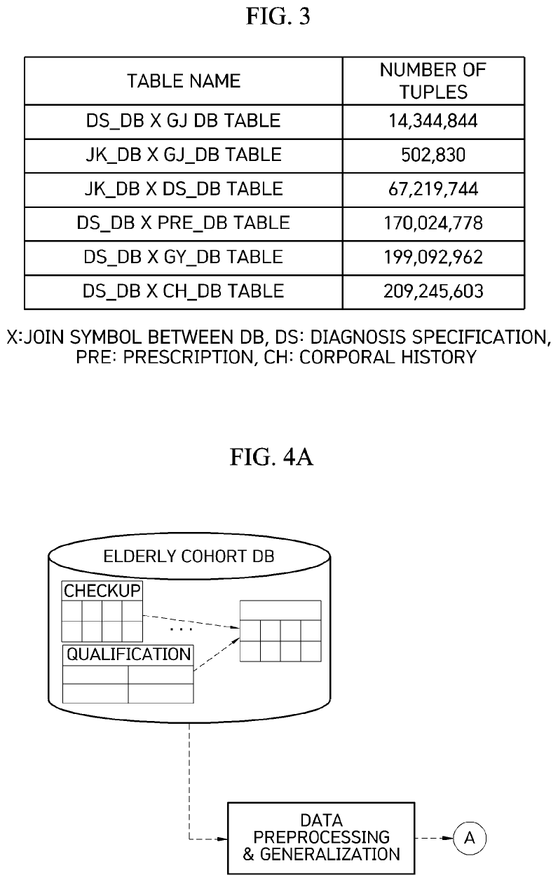

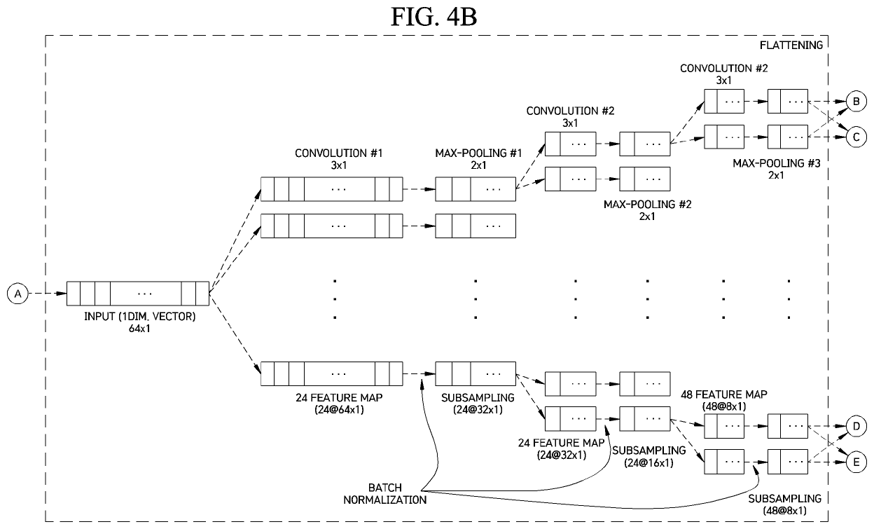

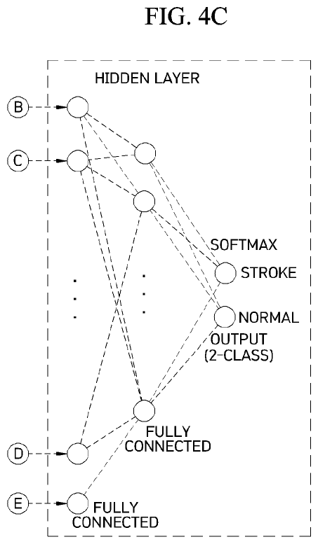

FIGS. 4a to 4c are reference diagram for describing the disease prediction model based on the 1D CNN according to the present invention.

Referring to FIGS. 4a to 4c, the disease prediction model according to the present invention may be constructed as the 1D CNN receiving cohort data of an elderly group and may include a convolution layer which extracts a feature of the cohort data preprocessed and input, a pooling layer, and a hidden layer for classifying the cohort data.

Moreover, referring to FIGS. 4a to 4c, the disease prediction model may include three convolution layers and three pooling layers, and moreover, may include two fully connected layers where all nodes are connected to one another. Here, the fully connected layer may be included in the hidden layer. A general CNN may perform modeling by stacking a plurality of fully connected layers, but the disease prediction model according to the present invention may have a difference in that only two fully connected layers are used.

Moreover, a softmax layer which evaluates a probability value associated with target disease prediction may be disposed at a final position of the hidden layer. For example, when the prediction target disease is cerebral stroke, the softmax layer may classify elderly persons having cerebral stroke and normal elderly persons and may classify elderly persons where an evaluated probability value is large.

Moreover, a rectified linear unit (ReLU) activation function may be used between each convolution layer and pooling layer of the disease prediction model, and a batch normalization process may be applied. Here, the ReLU activation function may be a function where a value less than 0 is returned as 0 and a value greater than 0 is returned as-is and may prevent slope disappearance which occurs when parameters are determined by adding the batch normalization process.

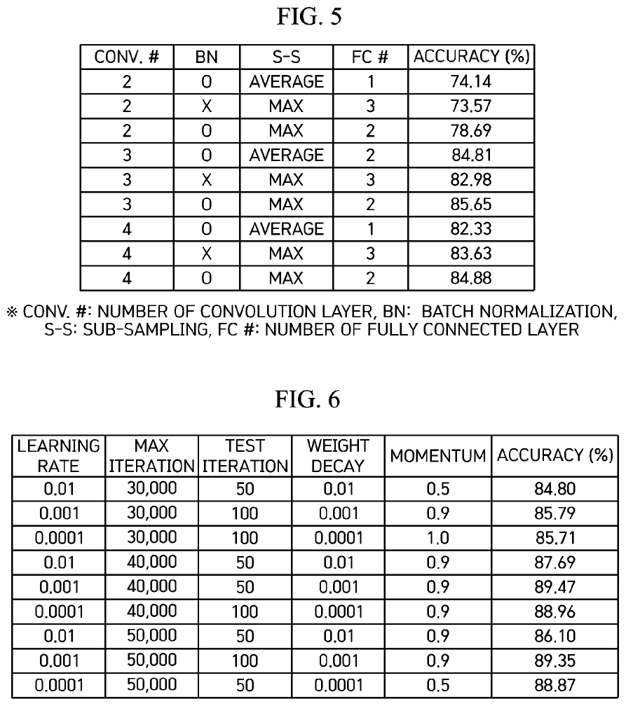

FIGS. 5 and 6 are reference tables showing an element-based analysis result of a disease prediction model according to an embodiment of the present invention.

Cohort data including data of 38,669 elderly persons having cerebral stroke and data of 38,669 normal elderly persons randomly extracted may be used for verifying the performance of the disease prediction apparatus 100 according to the present invention, and an experiment has been performed based on a data set of total 77,338 persons. In two kinds of experiments performed, 10-fold cross-validation has been applied, an optimizer has been applied to Adam, and hyper parameter tuning such as a learning rate and a performance number has been performed through changing as shown in a table of FIG. 6.

Referring to FIG. 5, in a first experiment, the number of convolution layers and the number of hidden layers have been differently set, and an experiment has been performed by changing the use or not of batch normalization and a sub sampling method. As a result of the experiment, three convolution layers and two fully connected layers have been used, and in sub sampling in the pooling layer, it has been confirmed that a disease prediction accuracy of cerebral stroke of elderly persons is highest in a case where max pooling and batch normalization are used.

Referring to FIG. 6, in a second experiment, the number of convolution layers and the number of hidden layers have been fixed, batch normalization has been used, and the disease prediction model has been analyzed by tuning a hyper parameter such as a learning rate and a performance number. Through the experiment, it has been confirmed that stable prediction performance is totally shown when a learning rate is 0.001 and a performance number is 40,000 or more.

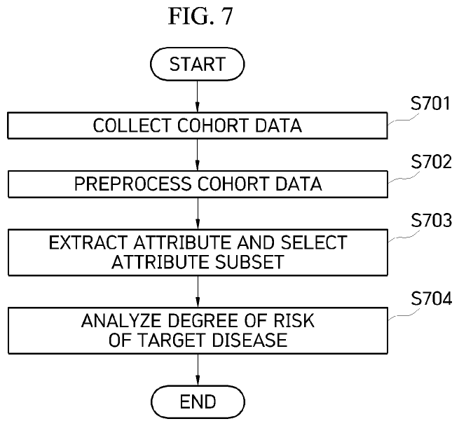

FIG. 7 is a flowchart for describing a process of predicting a disease by using elderly cohort data according to the present invention.

Referring to FIG. 7, cohort data of an elderly group may be collected, and for example, the cohort data of the elderly group may be collected in a research database which is built for research support for elderly persons such as prognosis analysis and risk factors of elderly diseases in step S701.

Subsequently, the cohort data may be preprocessed, and thus, may be processed into a format capable of being applied to a disease prediction model. Here, the data preprocessor 120 may remove a repeated tuple and a noise tuple in each data table included in the cohort data and may convert and normalize a data format so as to enable analysis through the disease prediction model in step S702.

Subsequently, the process may extract an attribute in the collected cohort data and may select a subset corresponding to the extracted attribute. Here, the extracted attribute may include a continuity attribute, including a body mass index, proteinuria, total cholesterol level, serum creatinine level, and gamma GPT level, and a discrete attribute including daily drinking amount, smoking, the presence of hepatitis B antigen (HBeAg), and high-strength physical activity in step S703.

Subsequently, the process may analyze the degree of risk of a target disease by using the selected subset. The degree of risk of the target disease may be determined based on a disease outbreak rate calculation result of the disease prediction model, and the disease prediction model may be constructed based on the 1D CNN in step S704.

In the above description, according to an implementation embodiment of the present invention, steps S701 to S704 may be further divided into additional steps, or may be combined as fewer steps. Also, some steps may be omitted depending on the case, and a sequence between steps may be changed. Furthermore, despite the other omitted content, the descriptions of FIGS. 1 to 6 may also be applied to FIG. 7.

An embodiment of the present invention described above may be implemented as a program (or an application) and may be stored in a medium, so as to be executed in connection with a server which is hardware.

The above-described program may include a code encoded as a computer language such as C, C++, JAVA, or machine language readable by a processor (CPU) of a computer through a device interface of the computer, so that the computer reads the program and executes the methods implemented as the program. Such a code may include a functional code associated with a function defining functions needed for executing the methods, and moreover, may include an execution procedure-related control code needed for executing the functions by using the processor of the computer on the basis of a predetermined procedure. Also, the code may further include additional information, needed for executing the functions by using the processor of the computer, or a memory reference-related code corresponding to a location (an address) of an internal or external memory of the computer, which is to be referred to by a media. Also, when the processor needs communication with a remote computer or server so as to execute the functions, the code may further include a communication-related code corresponding to a communication scheme needed for communication with the remote computer or server and information or a media to be transmitted or received in performing communication, by using a communication module of the computer.

The stored medium may denote a device-readable medium semi-permanently storing data, instead of a medium storing data for a short moment like a register, a cache, and a memory. In detail, examples of the stored medium may include read only memory (ROM), random access memory (RAM), CD-ROM, a magnetic tape, floppy disk, and an optical data storage device, but are not limited thereto. That is, the program may be stored in various recording mediums of various servers accessible by the computer or various recording mediums of the computer of a user. Also, the medium may be distributed to computer systems connected to one another over a network and may store a code readable by a computer in a distributed scheme.

The foregoing description of the present invention is for illustrative purposes, those with ordinary skill in the technical field of the present invention pertains in other specific forms without changing the technical idea or essential features of the present invention that may be modified to be able to understand. Therefore, the embodiments described above, exemplary in all respects and must understand that it is not limited. For example, each component may be distributed and carried out has been described as a monolithic and describes the components that are to be equally distributed in combined form, may be carried out.

The prevent invention may predict the outbreak of a disease on the basis of cohort data of an elderly group, and thus, may analyze the degree of risk of a target disease on the basis of all main risk factors.

The present invention may provide a risk analysis result of an elderly disease, thereby enabling medical facilities to easily provide objective diagnosis and a cure for a target disease.

The present invention may construct and apply a disease prediction model optimized for diseases of elderly persons of Korea to provide a high-accuracy analysis result of a target disease.

A number of exemplary embodiments have been described above.

Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A method of predicting disease by using elderly cohort data, the method comprising:
collecting cohort data of an elderly group;
preprocessing the collected cohort data;
extracting an attribute in the collected cohort data and selecting a subset corresponding to the extracted attribute; and
analyzing a degree of risk of a disease on the basis of the selected attribute set by using a disease prediction model.

2. The method of claim 1, wherein the preprocessing comprises generating a main data table associated with a disease which is to be predicted.

3. The method of claim 2, wherein the preprocessing comprises constructing a data mart including a data table associated with a main disease code of the disease which is to be predicted, on the basis of joining of the generated main data table.

4. The method of claim 1, wherein the collecting of the cohort data comprises:
periodically updating the cohort data stored in a database; and
previously teaching the disease prediction model on the basis of the updated cohort data of the database.

5. The method of claim 1, wherein the selecting of the subset comprises performing Z-score normalization based on the following Equation on the attribute extracted from the collected cohort data.
$$\vec{{x}_{i}} = \frac{{x}_{i} - μ}{σ} \times α$$
where xᵢ denotes each attribute, σ denotes a standard deviation of x, μ denotes an average of x, and α denotes a weight value.

6. The method of claim 1, wherein the selecting of the subset comprises selecting a subset of attributes extracted from the cohort data by using Hall's theorem.

7. The method of claim 1, wherein the selecting of the subset comprises evaluating a subset, where a largest value is calculated as a result of the calculation based on the following Equation, as a subset where an expression rate of all attributes is highest,
$$Merit ( {F}_{S} ) = \frac{k {r}_{cf}}{\sqrt{k + k ( k - 1 ) \overline{{r}_{ff}}}}$$
where Fₛ denotes a subset, k denotes the number of attributes of F_{z}, r̅_̅{̅c̅f̅}̅ denotes an average distribution of attributes included in Fₛ, and r̅_̅{̅f̅f̅}̅ denotes an average correlation value of all attributes.

8. A device for predicting disease by using elderly cohort data, the device comprising:
a data collector configured to collect cohort data of an elderly group;
a data preprocessor configured to preprocess the collected cohort data;
a subset selector configured to extract an attribute in the collected cohort data and select a subset corresponding to the extracted attribute; and
a disease analyzer configured to analyze a degree of risk of a disease on the basis of the selected attribute set by using a disease prediction model.

9. The device of claim 8, wherein the data collector periodically updating the cohort data stored in a database to previously teach the disease prediction model on the basis of the updated cohort data.

10. The device of claim 8, wherein the data preprocessor removes a repeated tuple and a noise tuple in each data table included in the cohort data and converts and normalizes a data format so as to enable analysis through the disease prediction model.

11. The device of claim 8, wherein the subset selector calculates and selects a subset where a probability distribution calculated in a case which uses all attributes extracted from the cohort data and a similar probability distribution are calculated, in performing data classification.

12. The device of claim 8, wherein the disease prediction model is constructed as a prediction model based on a 1D convolution neural network (CNN).

13. A method of generating a disease prediction model based on a 1D convolution neural network (CNN) structure by using cohort data of an elderly group, the method comprising:
placing a pooling layer and a convolution layer extracting a feature of the cohort data preprocessed and input; and
placing a hidden layer for classifying the cohort data.

14. The method of claim 13, wherein the placing of the pooling layer and the convolution layer comprises placing three convolution layers and three pooling layers.

15. The method of claim 13, wherein the placing of the hidden layer comprises placing two fully connected layers where all nodes are connected to one another.

16. The method of claim 13, wherein the placing of the hidden layer comprises placing a softmax layer which is disposed at a final position of the hidden layer and evaluates a probability value associated with target disease prediction.

17. The method of claim 13, wherein the placing of the pooling layer and the convolution layer comprises using a rectified linear unit (ReLU) activation function between each convolution layer and each pooling layer and applying batch normalization.
